(19) United States
(12) Patent Application Publication
(10) Pub. No.: US 2026/0069665 A1
(43) Pub. Date: Mar. 12, 2026

(54) SYSTEMS AND METHODS FOR EPICARDIAL TISSUE DISTENSION SOLUTIONS

(71) Applicant: Edwards Lifesciences Corporation, Irvine, CA (US)

(72) Inventor: Mazen Saadi Albaghdadi, Irvine, CA (US)

(21) Appl. No.: 19/386,948

(22) Filed: Nov. 12, 2025

Related U.S. Application Data
(63) Continuation of application No. PCT/US2024/029610, filed on May 16, 2024.
(60) Provisional application No. 63/502,914, filed on May 17, 2023.

(51) Int. Cl. A61K 38/46; A61K 31/575; A61K 31/685; A61K 38/48

(52) U.S. Cl. CPC A61K 38/465; A61K 31/575; A61K 31/685; A61K 38/486; A61K 38/4886; C12Y 301/01; C12Y 304/21011; C12Y 304/24

(57) ABSTRACT

Systems and methods for improving epicardial tissue distension are described. Drug delivery elements can deliver drugs that can dissolve epicardial fat and/or weaken pericardial connective tissue. Distension systems can provide mechanical distension of the pericardium. The drug delivery and the distension systems can be combined to improve epicardial tissue distension.

### CROSS-REFERENCE TO RELATED APPLICATIONS

The current application is a continuation of International Patent Application No. PCT/US2024/029610, filed May 16, 2024, which claims the benefit of U.S. Provisional Application No. 63/502,914, filed May 17, 2023, the disclosures of which are hereby incorporated by reference.

### FIELD OF THE DISCLOSURE

The present disclosure relates to systems and methods for epicardial tissue distension; more specifically, systems and methods for delivering drugs to create access space for epicardial tissue.

### BACKGROUND OF THE DISCLOSURE

Pericardial restraint is a normal physiologic process that may become exaggerated in some patients with heart failure with preserved ejection fraction (HFpEF), and cause the right heart to run out of space when filling, thereby squeezing and over pressurizing the left heart during physical activity in these patients. The increased left heart pressure can back up into the lungs and cause the patients to experience significant breathing difficulties when trying to do minimal activity. Increase in pericardial pressure can be caused by epicardial fat accumulation, which eventually expands the heart volume. Epicardial fat has been also associated with harmful metabolic effects which can affect functioning of the heart, coronary arteries, and microvasculature. Reduction of epicardial fat volume may improve heart functioning in HFpEF patients.

### SUMMARY OF THE DISCLOSURE

Systems and methods for improving epicardial tissue distension are described. Many examples implement drug delivery systems to deliver drugs that can break down epicardial fat accumulated around the epicardial tissue. Dissolved fat creates more space for epicardial tissue distension. In several examples, the drug delivery systems can deliver drugs that can break down the connective tissues and weaken the membranes of pericardium to allow epicardial tissue distension. In some examples, the drug delivery systems can be incorporated in an elastic ring that enhances the distension of epicardial tissue.

One example of the invention includes an epicardial tissue distension system, comprising: a plurality of drug delivery elements, wherein the plurality of drug delivery element is a dimensional element comprising a means for attachment to an epicardial tissue of the heart; and wherein the plurality of drug delivery elements comprises a lipolysis drug.

In another example, the plurality of drug delivery elements delivers a second drug to break down connective tissue of the pericardium to allow epicardial tissue distension.

In an additional example, the second drug is selected from the group consisting of a matrix metalloproteinase, a collagenase, and an elastase.

In a further example, at least one drug delivery element of the plurality of drug delivery elements has rounded corners.

In another further example, each drug delivery element of the plurality of drug delivery elements has a shape selected from the group consisting of: a sphere, a half sphere, a cube, a cuboid, a cylinder, a pylon, a cone, and any combinations thereof.

In an additional example again, each drug delivery element of the plurality of drug delivery elements has a maximum dimension from 5 mm to 2 cm.

In a further yet example, the lipolysis drug is: phosphatidylcholine, a lipase, or deoxycholic acid.

In another yet example, the plurality of drug delivery elements each comprises an attachment selected from the group consisting of: a barb, an anchor, a stick, a plug, an adhesive, and any combinations thereof.

In another example again, the plurality of drug delivery elements comprises a biodegradable polymer or a hydrogel.

In a further yet example again, the plurality of drug delivery elements comprises a polymer core encapsulating the drug to control the drug release rate.

Another further example comprises a distension ring that is capable of providing an outward force as it decompresses; wherein the plurality of drug delivery elements is located on the distension ring and faces outwardly against the pericardium.

In a yet further example, the plurality of drug delivery elements is attached on the distension ring via springs.

In an additional example again, the distension ring spans a portion or an entire circumference of the heart.

In a further yet example, the drug is released in a controlled manner.

In an additional example, at least one of the plurality of drug delivery elements comprises a bioresorbable material.

In yet another example, the drug is released by diffusion.

Another further example includes a method for improving epicardial tissue distension, comprising: installing a plurality of drug delivery elements on an epicardium of the heart, wherein the plurality of drug delivery elements is installed by attaching to the epicardium via a chemical means or a physical means; and wherein the plurality of drug delivery elements comprises a lipolysis drug.

In another example again, the plurality of drug delivery elements delivers a second drug to break down connective tissue of the pericardium to allow epicardial tissue distension.

In yet another example, the second drug is selected from the group consisting of a matrix metalloproteinase, a collagenase, and an elastase.

In a further yet example, the plurality of drug delivery elements has rounded corners.

In another further example, the plurality of drug delivery elements has a shape selected from the group consisting of: a sphere, a half sphere, a cube, a cuboid, a cylinder, a pylon, a cone, and any combinations thereof.

In yet another example, the plurality of drug delivery elements has a maximum dimension from 5 mm to 2 cm.

In a further example again, the lipolysis drug is selected from the group consisting of: phosphatidylcholine, lipase, and deoxycholic acid.

In yet another further example, the plurality of drug delivery elements each comprises an attachment selected from the group consisting of: a barb, an anchor, a stick, a plug, an adhesive, and any combinations thereof.

In a further example, the plurality of drug delivery elements comprises a biodegradable polymer or a hydrogel.

In another yet example, the plurality of drug delivery elements comprises a polymer core encapsulating the drug to control the drug release rate.

In a further yet example, the plurality of drug delivery elements is located on a distension ring that is capable of providing an outward force as it decompresses to allow epicardial tissue distension and the plurality of drug delivery elements faces outwardly against the pericardium.

In yet another example again, the plurality of drug delivery elements is attached on the distension ring via springs.

In an additional example, the distension ring spans a portion or an entire circumference of the heart.

In a further yet example, the drug is released in a controlled manner.

In yet another example, at least one of the plurality of drug delivery elements comprises a bioresorbable material.

In an additional example, the drug is released by diffusion.

Another example includes a method for drug delivery comprising: implanting a plurality of drug delivery elements between the epicardium and the pericardium of a heart; wherein the plurality of drug delivery elements is configured to deliver a drug to the epicardium, the pericardium, or a combination thereof.

In a further example, the drug comprises a lipolysis drug, a matrix metalloproteinase, or a combination thereof.

In an additional example, the plurality of drug delivery elements has rounded corners.

In yet another example, the plurality of drug delivery elements has a shape selected from the group consisting of: a sphere, a half sphere, a cube, a cuboid, a cylinder, a pylon, a cone, and any combinations thereof.

In a further yet example, the plurality of drug delivery elements has a maximum dimension from 5 mm to 2 cm.

In another example again, the plurality of drug delivery elements each comprises an attachment selected from the group consisting of: a barb, an anchor, a stick, a plug, an adhesive, and any combinations thereof.

In a further yet example again, the plurality of drug delivery elements comprises a biodegradable polymer or a hydrogel.

In an additional further example, the plurality of drug delivery elements comprises a polymer core encapsulating the drug to control the drug release rate.

In yet another example, the plurality of drug delivery elements is located on a distension ring that is capable of providing an outward force as it decompresses to allow epicardial tissue distension and the plurality of drug delivery elements faces outwardly against the pericardium.

In a further yet example, the plurality of drug delivery elements is attached on the distension ring via springs.

In an additional example, the distension ring spans a portion or an entire circumference of the heart.

In yet another example, the drug is released in a controlled manner.

In a further example again, at least one of the plurality of drug delivery elements comprises a bioresorbable material.

In a further yet example, the drug is released by diffusion.

Additional examples and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as examples of the invention and should not be construed as a complete recitation of the scope of the invention, wherein:

FIG. 1 conceptually illustrates a schematic of the heart walls.

FIG. 2A illustrates examples of a plurality of drug delivery elements implanted on the epicardium.

FIG. 2B illustrates examples of various structures of the drug delivery elements.

FIG. 3A illustrates examples of a drug delivery and distension system.

FIG. 3B illustrates examples of a side view of the drug delivery and distension system.

### DETAILED DESCRIPTION OF THE DISCLOSURE

Turning now to the drawings, systems and methods for improving access space in the pericardium of the heart are described. The pericardium surrounds myocardial tissue (i.e., muscular tissue) and comprises an epicardium tissue layer adjacent to the myocardial tissue, serous pericardium tissue layer that is a loose layer tissue that surround the heart, and the pericardial cavity, which is the space between the epicardial tissue and the pericardial tissue. For simplicity, throughout the disclosure, the term “pericardial tissue” refers to the serous pericardium tissue layer. In some individuals, the pericardium can begin to restrain the left ventricular muscle and hinder the ability for it to be filled. The inability of the left ventricle to properly fill overtime can be a significant contributor to left heart failure and related complications. Further, excess fat can accumulate in around the myocardial and epicardial tissues, resulting in a decrease of space within the pericardial cavity and an increase in the constricting pressure generated by the pericardial tissue. Expanding the space within the pericardial cavity will allow the epicardium to distend and thus alleviate pericardial tissue restraint, which can improve left ventricle filling to mitigate complications related to heart failure.

In order to alleviate pressures created by pericardial tissue, the various systems and methods of the disclosure create access space to allow for epicardial distension by breaking down fatty tissue accumulated within the epicardium. Drug delivery systems containing lipolysis drugs can be implanted in and around the epicardium. The lipolysis drugs can be released in a controlled manner or by diffusion around the epicardium to dissolve the epicardial fat. The dissolved fat enables epicardial tissue distension with the additional access space.

The pericardial tissue is formed of connective tissue including collagen and elastin. The pericardial tissue can stretch over time when subjected to gradual tensioning forces, for example due to excessive fluid accumulation. The drug delivery systems can deliver drugs to break down the pericardial connective tissue to create extra access space for epicardial distension. The drug molecules including (but not limited to) matrix metalloproteinases can be released in a controlled manner or by diffusion in and around the pericardial tissue. When the connective tissues are weakened by the drugs, the epicardium has more space to stretch.

Various drug delivery systems can be incorporated on an expandable ring to further assist epicardial distension. The ring, also referred as a distension ring, can be configured to press against the pericardial tissue to stretch it over time. The distension ring is capable of providing an outward force as it decompresses. The drug delivery systems can be located on the ring facing the pericardial tissue. The combined drug delivery systems and the distension ring can be implanted between the myocardium and the pericardial tissue. A plurality of the drug delivery systems located on the ring can release drugs including the lipolysis drugs to break down epicardial fat and/or the matrix metalloproteinases to break down the connective tissue.

The drug delivery systems can have various structures and geometries. Some drug delivery systems implement catheters to deliver drug molecules. The drug delivery catheters can be advanced in a subxiphoid approach toward and through the pericardial tissue to release drugs configured to break down epicardial fat. Several drug delivery systems comprise a plurality of drug delivery elements. The plurality drug delivery elements can comprise a variety of shapes including (but not limited to) spheres, half spheres, cubes, cylinders, cuboids, cones, tetrahedrons, hexagons, octahedrons, ellipsoids, pyramids, pylons, and any combinations thereof. Some systems combine half spheres and cuboids; or half spheres and cubes; or elliptical rings and cuboids; or elliptical rings and cubes. In order to avoid puncture and better distribute distension forces, the drug delivery systems have rounded corners instead of sharp ones.

The plurality of drug delivery elements of the drug delivery systems can vary in sizes. A maximum dimension of a drug delivery element ranges from about 5 mm to about 2 cm. The spheres and/or the half spheres can have a diameter from about 5 mm to about 2 cm. The cubes can have a length or an edge from about 5 mm to about 2 cm. The cylinders can have a height from about 5 mm to about 2 cm, and a diameter less than 2 cm. The cylinders can have a diameter from about 5 mm to about 2 cm, and a height less than 2 cm. The cuboids can have a length from about 5 mm to about 2 cm, a height less than about 2 cm, and a width less than about 2 cm.

The plurality of drug delivery elements of the drug delivery systems can be implanted on the epicardium using various attachment mechanisms. The attachment can be achieved via physical means and/or chemical means. Various chemical means for attachment include (but are not limited to) forming chemical bonds (hydrogen bonds, covalent bonds), crosslinking, via static charge, and any combinations thereof. Various physical means for attachment include (but are not limited to) barbs, anchors, sticks, plugs, adhesives, and any combinations thereof. Certain drug delivery systems comprising a plurality of drug delivery elements can be attached to the distension ring via springs and/or elastics. The drug delivery elements can be angled to better engage with the pericardium via the springs and/or elastics. The springs and/or elastics also provide additional support for distension.

Systems for epicardial tissue distension can expand the epicardium in a symmetrical manner. The distension systems can provide expansion in a range less than about 2 cm; or from about 1 cm to about 2 cm; or less than about 1 cm. The epicardial tissue can be distended outwardly towards the pericardial tissue. The distension ring can provide a radial distension force.

The drug delivery systems can be made of biocompatible and/or bioresorbable polymers. The drug molecules encapsulated in bioresorbable polymers can be slowly released when the polymer resorbs overtime. Examples of bioresorbable polymers include (but are not limited to) PLA and PLGA. Some drug delivery systems can be made of hard materials such as biocompatible plastics. A number of examples implement hydrogels for the drug delivery systems. The distension ring can be made of a material that is expandable or elastic, biocompatible, and/or biodegradable. Examples of such materials include (but are not limited to) PLA and PLGA.

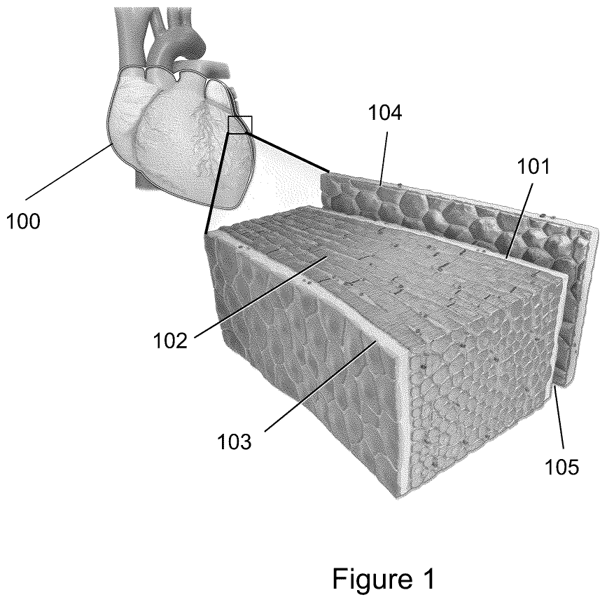

FIG. 1 conceptually illustrates a schematic of the heart wall. The heart wall 100 can include three distinctive layers: the endocardium, myocardium, and epicardium. The epicardium 101 refers to the outermost protective layer of the heart and is considered part of the pericardium. The epicardium is composed of mesothelium, a layer of cells that covers and protects most of the internal organs of the body as well as fat and connective tissue. The epicardium predominantly surrounds the heart and the roots of the coronary vessels emerging from it, including the aorta, the superior vena cava, and inferior vena cava. The other two layers of the heart wall include the myocardium 102 (i.e., the middle muscular layer that supports the pumping capabilities of the heart) and the endocardium 103, or the innermost layer. Epicardial fat (not shown) is the adipose tissue located between the epicardium 101 and the myocardium 102. The pericardium, also known as pericardial sac, is a double-walled sac containing the heart and the roots of the great vessels. The pericardium includes the epicardium 101, the serous pericardial tissue 104 and the pericardial cavity 105, which contains pericardial fluid. Pericardial tissue 104 separates the heart from interference of other structures and provides protection. The pericardium is a tough fibroelastic sac which covers the heart from almost all sides.

### Drug Delivery Systems for Epicardial Tissue

Systems and methods for improving the ability for epicardial tissue to distend are described. Drug delivery systems can be implanted on the epicardial tissue to deliver drugs that can break down the epicardial fat accumulated around the epicardial tissue. The drug delivery systems can include a drug delivery catheter to deliver the drugs. The drugs can be delivered via a catheter system employing an injection method. The drug delivery catheter can be advanced via a subxiphoid approach toward and through the pericardial tissue to deliver drugs that can break down and reduce the volume of epicardial fat. The drug delivery catheter can be implanted during a process including (but not limited to) pericardiocentesis procedures. During the pericardiocentesis procedures, a needle can be inserted into the pericardium, through which a guidewire can be passed. Once the guidewire is placed, the needle can be retrieved. The drug delivery catheter can then be advanced over the guidewire. The distal end of the catheter can contain the drugs for breaking down fat. Controlling the proximal end of the catheter can release the drug into the target tissue. Examples of the drugs include (but are not limited to) drugs for breaking down subcutaneous fat or lipolysis drugs. Examples of the drugs include (but are not limited to) phosphatidylcholine, lipase, and deoxycholic acid. As can readily be appreciated, any of a variety of fat dissolving drugs can be utilized as appropriate to the requirements of specific applications in accordance with various embodiments of the invention.

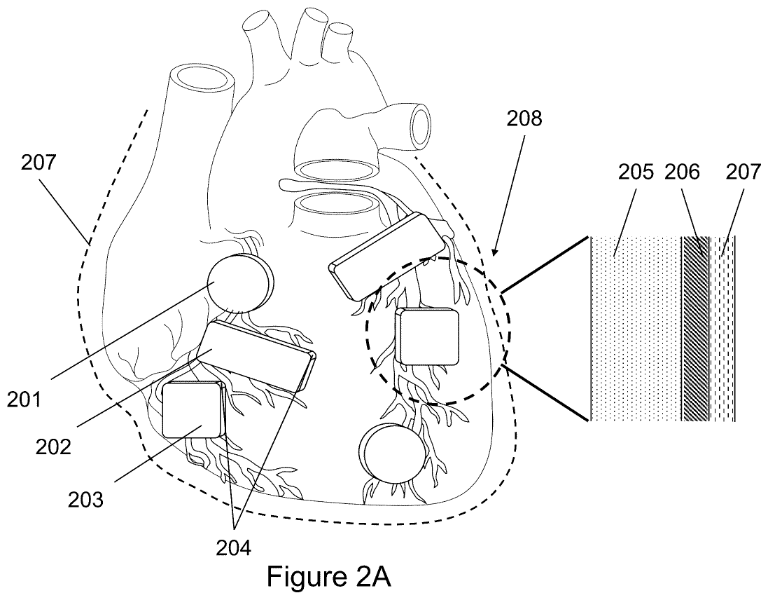

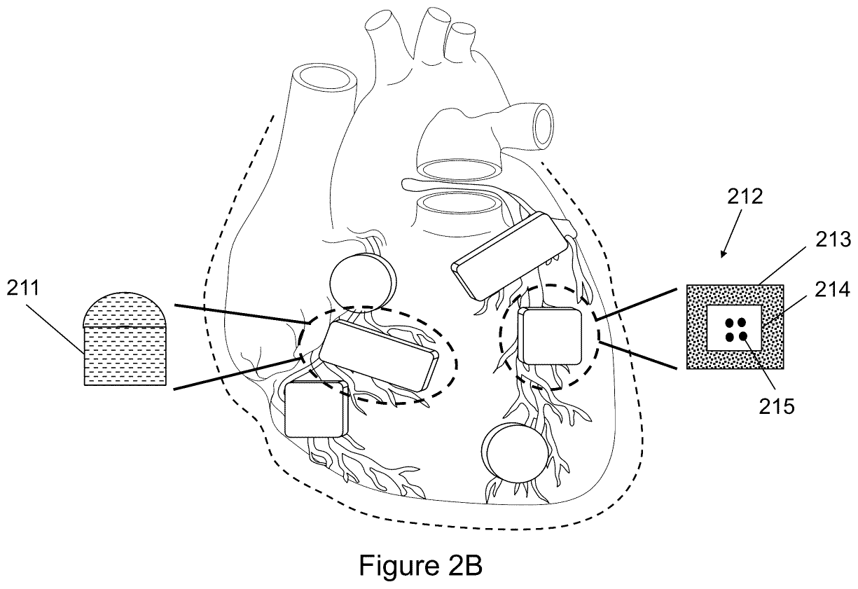

The drug delivery systems include one or more drug delivery elements that can be implanted on the epicardial tissue. FIG. 2A illustrates a plurality of drug delivery elements implanted on the epicardium in accordance with an example. The drug delivery elements 201, 202, 203 can be installed at desired locations (such as near regions of fat accumulation) on the epicardial tissue and may be positioned to avoid compression of the coronary arteries, or be used to directly administer therapies to the underlying coronary arteries. The drug delivery elements can be arranged in a row; or in parallel; or in a symmetrical pattern; or in an asymmetrical pattern. The implanted one or more drug delivery elements can have the same shapes or different shapes. Various shapes for the drug delivery elements include (but are not limited to) cylinders 201, cuboids 202, cubes 203, spheres (not shown), half spheres (not shown), and any combinations thereof. Specific shapes of the drug delivery elements can be chosen depending on the anatomy and distribution of epicardial fat. The drug delivery elements have rounded corners 204 to prevent punctures. The drug delivery elements 201, 202, 203 can have various attachment mechanisms to be attached to the epicardium. Attachment can be performed by physical means (such as an anchor) and/or chemical means (such as a biocompatible adhesive). Physical elements such as barbs, anchors, sticks, plugs, adhesives, and any combinations thereof, can be attached to one side of the device in order to land on the epicardium. Chemical mechanisms including (but not limited to) forming hydrogen bonds, forming covalent bonds, crosslinking, via static charge, and any combinations thereof, can also be applied for attaching the devices on the epicardium.

The drug delivery systems can vary in sizes. The cylindrical drug delivery elements 201 can have a height from about 5 mm to about 2 cm, and a diameter less than 2 cm; or a diameter from about 5 mm to about 2 cm, and a height less than 2 cm. The cuboids 202 can have a length from about 5 mm to about 2 cm, a height less than about 2 cm, and a width less than about 2 cm. The cubical devices 203 can have a length from about 5 mm to about 2 cm.

An enlarged illustration 208 of a drug delivery element shows that device 206 can be installed in the pericardial cavity (also known as the pericardial space or the pericardial sac), between the epicardium 205 and the pericardial tissue 207. The devices 206 can deliver drugs that can break down the accumulated epicardial fat and/or drugs that can break down the connective tissue of the pericardial tissue. The fat dissolving drugs can traverse or disperse through the epicardial tissue 205 to break down the fat between the epicardium and the myocardium. Dissolved fat increases the space for the epicardium to distend. Examples of drugs that can break down fat include (but are not limited to) drugs for dissolving subcutaneous fat, lipolysis drugs, Phosphatidylcholine, Lipase, and Deoxycholic acid, which break down the fat into its chemical components and can be reabsorbed by the body. Pericardial tissue 207 surrounding the epicardium, comprises connective tissues made of collagen and elastin. Delivering drugs to digest or weaken the connective tissues can release more space for epicardial tissue distension. Examples of drugs that can weaken the connective tissue include (but are not limited to) matrix metalloproteinases, collagenase, and elastase. As can readily be appreciated, any of a variety of drugs that can break down the connective tissues can be utilized as appropriate to the requirements of specific applications to be performed.

The additional space created by the dissolved fat tissue and/or the dissolved connective tissue enables the ability of epicardial tissue to expand. The systems can provide expansion space in a range of less than about 2 cm; or from about 1 cm to about 2 cm; or less than about 1 cm.

FIG. 2B illustrates various structures of the drug delivery elements. 211 shows a cross section view of a drug delivery element with a half sphere positioned on top of a cuboid. The rounded surface design can reduce point forces and limit puncture. The drug delivery elements can release the drugs in a controlled manner or by diffusion. In some examples, the devices can be designed for long-term release where the outer layer of the device comprise biodegradable polymers and the inner layer comprises a polymer core encapsulating the drugs. As the biodegradable polymers dissolve, the encapsulated drugs can be slowly release into the tissues. 212 shows a cross section view of a drug delivery element for long term release. The outer layer of the device 213 can be made of a biodegradable and biocompatible material, with an inner polymeric core 214 containing desired drug molecules 215. The inner and outer layer design can further control the drug release rate.

While various systems for drug delivery systems for epicardial distension are described above with reference to FIGS. 2A and 2B, any variety of systems that utilize drug delivery systems to enhance epicardial distension can be utilized as appropriate to the requirements of specific applications to be performed.

### Drug Delivery and Distension Systems

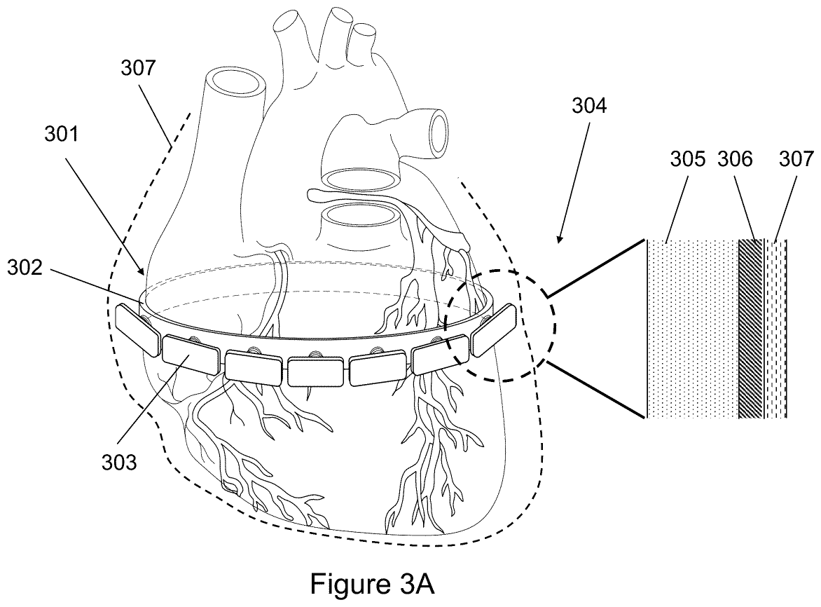

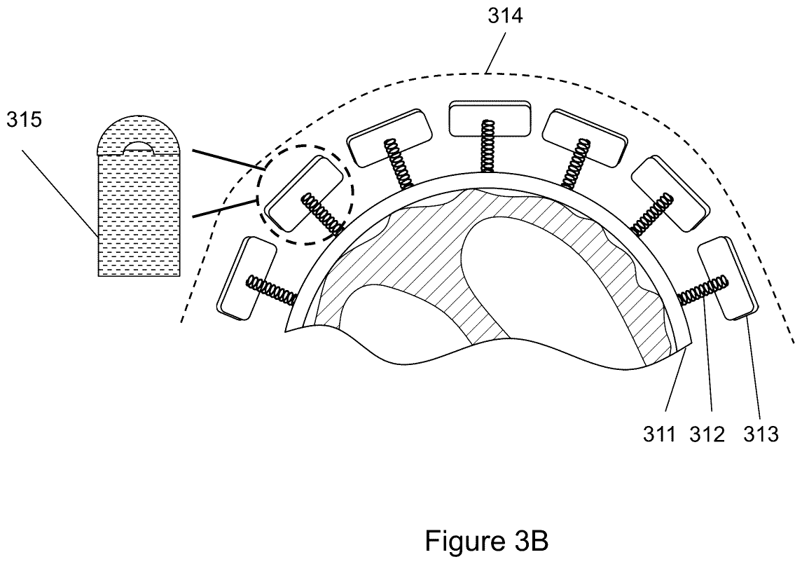

In many examples, a drug delivery system comprising a plurality of drug delivery elements can be incorporated on a distension ring. The distension ring can be installed between the epicardium and the pericardium and include a plurality of drug delivery elements configured to press against the pericardium to stretch it over time. The distension ring can provide gradual mechanical distension on pericardial tissue. The ring and/or the drug delivery elements can be designed to release drugs. The released drugs can break down the connective tissue toward the pericardium, and/or break down epicardial fat toward the epicardium. FIG. 3A illustrates a drug delivery and distension system in accordance with an example. The drug delivery and distension system 301 includes a support ring 302, which can span the entire circumference of the heart or only a portion of its circumference. The ring 302 can be disposed in the pericardial space between the epicardium and the pericardium. A plurality of drug delivery elements 303 can be attached to the ring 302 and may be stacked over each other, having their free ends biased radially against the pericardium.

An enlargement of a portion of the drug delivery and distension system 304 shows that the system 306 can be implanted in the pericardial cavity, between the epicardium 305 and the pericardial tissue 307. The distension ring and/or the biasing elements can deliver drugs that can break down the epicardial fat and/or drugs that can break down the connective tissue of the pericardial tissue. Dissolved fat tissue releases the distension space for the epicardium. Examples of drugs that can break down fat include (but are not limited to) lipolysis drugs, such as phosphatidylcholine, lipase, and deoxycholic acid. The pericardium is a distensible tissue. The biasing elements of the distension system can face and push towards the pericardium to create distension space. In addition, drugs that can digest or weaken the connective tissues can release more space for epicardial tissue distension. Examples of drugs can include (but are not limited to) pharmaceutical compositions that comprise one or more of matrix metalloproteinases, collagenase, and elastase.

FIG. 3B illustrates a side view of the drug delivery and distension system in accordance with an example. The distension ring 311 with outwardly biased drug delivery elements 313 can be positioned on the epicardium and facing the pericardial tissue 314. The angle of engagement of the drug delivery elements 313 with the pericardial tissue 314 may differ according to heart-pericardial orientation. The drug delivery elements 313 can have various shapes including (but not limited to) pylons or cylinders. 315 shows a side view of the drug delivery element where a rounded cap is positioned on a cuboid to better distribute the distension force acting against the pericardial tissue. The drug delivery elements 313 can be supported on springs or elastics 312. The springs or elastics 312 can be compressed springs or compressed elastics. The springs can provide more distension forces towards the pericardium. As the pericardial tissue stretches over time, the drug delivery elements supported on the springs extend further outward to further distend the pericardial tissue. The ring can be formed to release drugs toward the epicardial surface, and the drug delivery elements can similarly release drugs that digest or weaken the pericardial over time, such as collagenase or elastase.

While various systems for combining drug delivery and distension are described above with reference to FIGS. 3A and 3B, any variety of systems that utilize drug delivery systems and distension rings to improve epicardial distension can be utilized as appropriate to the requirements of specific applications to be performed.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present, or problems be solved.

Various examples of systems and components for epicardial distension thereof are disclosed herein, and any combination of these examples can be made unless specifically excluded. For example, drug delivery elements of various structures and sizes can be utilized, even if a specific combination is not explicitly described. Likewise, the different constructions and features of drug delivery systems and the distension ring can be mixed and matched, such as by combining the various components of the systems, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

Various systems and devices for drug delivery are utilized for the purpose of distending epicardium within a recipient. Recipients include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms.

The described systems and devices can be sterilized, which can be performed using gamma irradiation, gas plasma, aldehydes, ethylene oxide, and/or e-beam. The systems or devices can be further treated with a formaldehyde bioburden reduction process. After preparation, the systems and devices can be stored within a container, which can be hermetically sealed or otherwise kept sterile.

### Doctrine of Equivalents

For purposes of this description, certain aspects, advantages, and novel features of the instances of this disclosure are described herein. The disclosed methods, apparatus, and systems should not be construed as being limiting in any way. Instead, the present disclosure is directed toward all novel and nonobvious features and aspects of the various disclosed instances, alone and in various combinations and sub-combinations with one another. The methods, apparatus, and systems are not limited to any specific aspect or feature or combination thereof, nor do the disclosed instances require that any one or more specific advantages be present, or problems be solved.

Although the operations of some of the disclosed instances are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises. ” Reference to an object in the singular is not intended to mean “one and only one” unless explicitly so stated, but rather “one or more. ” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1 %, less than or equal to ±0.5%, less than or equal to ±0.1 %, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In view of the many possible instances to which the principles of the disclosed technology may be applied, it should be recognized that the illustrated instances are only preferred examples and should not be taken as limiting the scope of the disclosure. Rather, the scope of the disclosure is at least as broad as the following claims.

### EXAMPLES

### Example 1

An epicardial tissue distension system, comprising: a plurality of drug delivery elements, wherein the plurality of drug delivery element is a dimensional element comprising a means for attachment to an epicardial tissue of the heart; and wherein the plurality of drug delivery elements comprises a lipolysis drug.

### Example 2

The system of example 1, wherein the plurality of drug delivery elements delivers a second drug to break down connective tissue of the pericardium to allow epicardial tissue distension.

### Example 3

The system of example 1 or 2, wherein the second drug is selected from the group consisting of a matrix metalloproteinase, a collagenase, and an elastase.

### Example 4

The system of examples 1, 2, or 3, wherein at least one drug delivery element of the plurality of drug delivery elements has rounded corners.

### Example 5

The system of any one of examples 1-4, wherein each drug delivery element of the plurality of drug delivery elements has a shape selected from the group consisting of: a sphere, a half sphere, a cube, a cuboid, a cylinder, a pylon, a cone, and any combinations thereof.

### Example 6

The system of any one of examples 1-5, wherein each drug delivery element of the plurality of drug delivery elements has a maximum dimension from 5 mm to 2 cm.

### Example 7

The system of any one of examples 1-6, wherein the lipolysis drug is: phosphatidylcholine, a lipase, or deoxycholic acid.

### Example 8

The system of any one of examples 1-7, wherein the plurality of drug delivery elements each comprises an attachment selected from the group consisting of: a barb, an anchor, a stick, a plug, an adhesive, and any combinations thereof.

### Example 9

The system of any one of examples 1-8, wherein the plurality of drug delivery elements comprises a biodegradable polymer or a hydrogel.

### Example 10

The system of any one of examples 1-9, wherein the plurality of drug delivery elements comprises a polymer core encapsulating the drug to control the drug release rate.

### Example 11

The system of any one of examples 1-10, further comprising a distension ring that is capable of providing an outward force as it decompresses; wherein the plurality of drug delivery elements is located on the distension ring and faces outwardly against the pericardium.

### Example 12

The system of any one of examples 1-11, wherein the plurality of drug delivery elements is attached on the distension ring via springs.

### Example 13

The system of any one of examples 1-12, wherein the distension ring spans a portion or an entire circumference of the heart.

### Example 14

The system of any one of examples 1-13, wherein the drug is released in a controlled manner.

### Example 15

The system of any one of examples 1-14, wherein at least one of the plurality of drug delivery elements comprises a bioresorbable material.

### Example 16

The system of any one of examples 1-15, wherein the drug is released by diffusion.

### Example 17

A method for improving epicardial tissue distension, comprising: installing a plurality of drug delivery elements on an epicardium of the heart, wherein the plurality of drug delivery elements is installed by attaching to the epicardium via a chemical means or a physical means; and wherein the plurality of drug delivery elements comprises a lipolysis drug.

### Example 18

The method of example 17, wherein the plurality of drug delivery elements delivers a second drug to break down connective tissue of the pericardium to allow epicardial tissue distension.

### Example 19

The method of example 17 or 18, wherein the second drug is selected from the group consisting of a matrix metalloproteinase, a collagenase, and an elastase.

### Example 20

The method of example 17, 18, or 19, wherein the plurality of drug delivery elements has rounded corners.

### Example 21

The system of any one of examples 17-20, wherein the plurality of drug delivery elements has a shape selected from the group consisting of: a sphere, a half sphere, a cube, a cuboid, a cylinder, a pylon, a cone, and any combinations thereof.

### Example 22

The system of any one of examples 17-21, wherein the plurality of drug delivery elements has a maximum dimension from 5 mm to 2 cm.

### Example 23

The system of any one of examples 17-22, wherein the lipolysis drug is selected from the group consisting of: phosphatidylcholine, lipase, and deoxycholic acid.

### Example 24

The system of any one of examples 17-23, wherein the plurality of drug delivery elements each comprises an attachment selected from the group consisting of: a barb, an anchor, a stick, a plug, an adhesive, and any combinations thereof.

### Example 25

The system of any one of examples 17-24, wherein the plurality of drug delivery elements comprises a biodegradable polymer or a hydrogel.

### Example 26

The system of any one of examples 17-25, wherein the plurality of drug delivery elements comprises a polymer core encapsulating the drug to control the drug release rate.

### Example 27

The system of any one of examples 17-26, wherein the plurality of drug delivery elements is located on a distension ring that is capable of providing an outward force as it decompresses to allow epicardial tissue distension and the plurality of drug delivery elements faces outwardly against the pericardium.

### Example 28

The system of any one of examples 17-27, wherein the plurality of drug delivery elements is attached on the distension ring via springs.

### Example 29

The system of any one of examples 17-28, wherein the distension ring spans a portion or an entire circumference of the heart.

### Example 30

The system of any one of examples 17-29, wherein the drug is released in a controlled manner.

### Example 31

The system of any one of examples 17-30, wherein at least one of the plurality of drug delivery elements comprises a bioresorbable material.

### Example 32

The system of any one of examples 17-31, wherein the drug is released by diffusion.

### Example 33

A method for drug delivery, comprising: implanting a plurality of drug delivery elements between the epicardium and the pericardium of a heart; wherein the plurality of drug delivery elements is configured to deliver a drug to the epicardium, the pericardium, or a combination thereof.

### Example 34

The method of example 33, wherein the drug comprises a lipolysis drug, a matrix metalloproteinase, or a combination thereof.

### Example 35

The method of example 33 or 34, wherein the plurality of drug delivery elements has rounded corners.

### Example 36

The method of example 33, 34, or 35, wherein the plurality of drug delivery elements has a shape selected from the group consisting of: a sphere, a half sphere, a cube, a cuboid, a cylinder, a pylon, a cone, and any combinations thereof.

### Example 37

The method of any one of examples 33-36, wherein the plurality of drug delivery elements has a maximum dimension from 5 mm to 2 cm.

### Example 38

The method of any one of examples 33-37, wherein the plurality of drug delivery elements each comprises an attachment selected from the group consisting of: a barb, an anchor, a stick, a plug, an adhesive, and any combinations thereof.

### Example 39

The method of any one of examples 33-38, wherein the plurality of drug delivery elements comprises a biodegradable polymer or a hydrogel.

### Example 40

The method of any one of examples 33-39, wherein the plurality of drug delivery elements comprises a polymer core encapsulating the drug to control the drug release rate.

### Example 41

The method of any one of examples 33-40, wherein the plurality of drug delivery elements is located on a distension ring that is capable of providing an outward force as it decompresses to allow epicardial tissue distension and the plurality of drug delivery elements faces outwardly against the pericardium.

### Example 42

The method of any one of examples 33-41, wherein the plurality of drug delivery elements is attached on the distension ring via springs.

### Example 43

The method of any one of examples 33-42, wherein the distension ring spans a portion or an entire circumference of the heart.

### Example 44

The method of any one of examples 33-43, wherein the drug is released in a controlled manner.

### Example 45

The method of any one of examples 33-44, wherein at least one of the plurality of drug delivery elements comprises a bioresorbable material.

### Example 46

The method of any one of examples 33-45, wherein the drug is released by diffusion.

1. A system for improving epicardial tissue distension, comprising:
a plurality of drug delivery elements configured for attachment to epicardial tissue of a heart;
wherein the plurality of drug delivery elements comprises a lipolysis drug for breaking down epicardial fat.

2. The system of claim 1, wherein the plurality of drug delivery elements delivers a second drug for breaking down connective tissue of a pericardium.

3. The system of claim 2, wherein the second drug is selected from the group consisting of a matrix metalloproteinase, a collagenase, and an elastase.

4. The system of claim 1, wherein the system is capable of improving left ventricle filling and mitigates complications related to heart failure.

5. The system of claim 1, wherein each drug delivery element of the plurality of drug delivery elements has a shape selected from the group consisting of a sphere, a half sphere, a cube, a cuboid, a cylinder, a pylon, a cone, and any combinations thereof.

6. The system of claim 1, wherein each drug delivery element of the plurality of drug delivery elements has a maximum dimension of 2 cm.

7. The system of claim 1, wherein the lipolysis drug is phosphatidylcholine, a lipase, or deoxycholic acid.

8. The system of claim 1, wherein the plurality of drug delivery elements each comprises an attachment feature selected from the group consisting of a barb, an anchor, a stick, a plug, an adhesive, or any combinations thereof.

9. The system of claim 1, wherein the plurality of drug delivery elements comprises a biodegradable polymer or a hydrogel.

10. The system of claim 1, wherein the plurality of drug delivery elements comprises a polymer core encapsulating the drug to control a drug release rate.

11. The system of claim 1, further comprising a distension ring for providing an outward force, wherein the plurality of drug delivery elements is located on the distension ring and faces outwardly against an inner wall of pericardium.

12. The system of claim 1, wherein at least one of the plurality of drug delivery elements comprises a bioresorbable material.

13. A system for improving heart function by enhancing epicardial tissue distension, the
system comprising:
an elongate catheter; and
a drug delivery element coupled to a distal end portion of the elongate catheter, wherein the drug delivery element includes an anchoring feature for attachment along a surface of epicardial tissue of a heart;
wherein the drug delivery element releases a lipolysis drug after attachment to the epicardial tissue.

14. The system of claim 13, wherein the drug delivery element releases a second drug to reduce connective tissue in pericardial tissue, wherein the second drug is selected from the group consisting of a matrix metalloproteinase, a collagenase, and an elastase.

15. The system of claim 14, wherein the lipolysis drug is selected from the group consisting of phosphatidylcholine, lipase, and deoxycholic acid. a matrix metalloproteinase, a collagenase, and an elastase.

16. The system of claim 15, wherein the second drug is selected from the group consisting of phosphatidylcholine, lipase, and deoxycholic acid.

17. A system for mitigating complications related to heart failure by allowing enhanced
epicardial tissue distension, the system comprising:
an elongate catheter;
a drug delivery element coupled to a distal end portion of the elongate catheter, wherein the drug delivery element includes an anchoring feature for attachment to a surface of epicardial tissue in a heart, wherein the drug delivery element controllably releases a lipolysis drug into the heart for dissolving epicardial fat; and
a pushing mechanism shaped for deployment in an epicardial space for providing an outward force on a pericardial wall.

18. The system of claim 17, wherein the drug delivery element comprises a biodegradable material that encapsulates at least of portion of the lipolysis drug for allowing the controllable release.

19. The system of claim 18, wherein the biodegradable material is a polymer.

20. The system of claim 18, wherein the biodegradable material is a hydrogel.
